(19) United States
(12) Patent Application Publication
(10) Pub. No.: US 2010/0087861 A1
(43) Pub. Date: Apr. 8, 2010

(54) BONE FIXATION ELEMENT

(75) Inventors: Beat Lechmann, Grenchen (CH); Roger Buerki, Chur (CH)

Correspondence Address: STROOCK & STROOCK & LAVAN, LLP, 180 MAIDEN LANE, NEW YORK, NY, 10038, US

(73) Assignee: SYNTHES (U.S.A.), West Chester, PA (US)

(21) Appl. No.: 12/529,691

(22) PCT Filed: Apr. 9, 2008
(86) PCT No.: PCT/US2008/059758

§ 371 Date: Sep. 2, 2009

Related U.S. Application Data
(60) Provisional application No. 60/910,758, filed on Apr. 9, 2007.

(51) Int. Cl. A61B 17/70; A61B 17/86

(52) U.S. Cl. 606/257; 606/302

(57) ABSTRACT

A bone fixation element (10) for use in spinal fixation facilitates insertion of a longitudinal spinal rod (45) in a rod-receiving channel (26) formed in the bone fixation element. The bone fixation element engages a coated spinal rod, preferably a dynamic spinal rod made from a generally non-biocompatible material such as nickel, cobalt chromium or Nitinol. The bone fixation element preferably incorporates first (120) and second (140) rod protectors to contact the coating on the spinal rod when the rod is received in the rod receiving channel of the hone fixation element. The first and second rod protectors are preferably constructed of a material having a hardness that is less than a hardness of a material of the coated spinal rod.

### CROSS-REFERENCE TO RELATED APPLICATIONS

This application claims priority to U.S. provisional patent application Ser. No. 60/910,758, filed Apr. 9, 2007, the entire content of which is hereby incorporated by reference in its entirety.

### BACKGROUND OF THE INVENTION

It is often necessary due to various spinal disorders to surgically correct and stabilize spinal curvatures, or to facilitate spinal fusion. Numerous systems for treating spinal disorders have been disclosed. One known method involves a pair of elongated members, typically relatively rigid spinal rods, longitudinally placed on the posterior spine on either side of spinous processes of the vertebral column. Each rod is attached to two or more vertebrae along the length of the spine by way of vertebra engaging bone fixation elements. The bone fixation elements commonly include a body portion incorporating a rod-receiving channel for receiving the longitudinal spinal rod therein. Moreover, the body portion often includes a mechanism for receiving a closure cap to clamp and fix the position of the spinal rod with respect to the bone fixation element.

Recently, dynamic spinal rods (e.g., bendable) have been utilized in spinal surgery. Dynamic spinal rods may absorb shock, for example, in the extension and compression of the spine. Treatment using a dynamic spinal rod may not provide dampening along the longitudinal axis of the rod. However, the dynamic spinal rod may be bendable in order to preserve the mobility of the spinal segment. Dynamic spinal rods may be formed from generally non-biocompatible materials to enhance their bendability. To enhance the biocompatibility of these dynamic spinal rods, the rods may be coated to improve the material properties of the rods, and/or for other reasons.

If the body portion of the bone fixation element to which the dynamic spinal rod is connected is made from a metal, such as, for example, titanium or a titanium alloy, it is possible that contact between the body portion of the bone fixation element and the coated rod may damage the rod's coating, especially if there is a high level of stress between the two components.

### BRIEF SUMMARY OF THE INVENTION

The present application is directed to a bone fixation element for use in spinal fixation to facilitate insertion of a longitudinal spinal rod in a rod-receiving channel formed in the bone fixation element. More preferably, the present application is directed to a bone fixation element for use with a coated dynamic spinal rod preferably constructed from a generally non-biocompatible material such as, for example, nickel, a nickel alloy such as Ni—Ti-Alloy (e.g., Nitinol), cobalt chromium, cobalt chromium alloy, etc. The bone fixation element preferably incorporates first and second rod protectors to help preserve the integrity of the coating on the spinal rod when the rod is received in the rod receiving channel of the bone fixation element. The first and second rod protectors preferably are made from a softer material when compared to the coated spinal rod.

In one exemplary embodiment, the bone fixation system may include a coated longitudinal rod and at least two bone fixation elements, wherein each bone fixation element includes a bone anchor for securing the bone fixation element to a patient's bone such as, for example, a vertebra. A body portion has an inner bore and a rod-receiving channel dimensioned to receive the coated longitudinal rod. A first rod protector is dimensioned to fit within the inner bore of the body portion and the first rod protector has a top surface for contacting the coated rod. A second rod protector is dimensioned to fit within the inner bore of the body and the second rod protector has a bottom surface for contacting the coated rod. A closure cap is configured to engage the body portion for at least partially obstructing the rod receiving channel to prevent the coated rod from escaping from the body portion. The first and second rod protectors are preferably made from a softer material when compared to the coated spinal rod.

### BRIEF DESCRIPTION OF THE SEVERAL VIEWS OF THE DRAWINGS

The foregoing summary, as well as the following detailed description of the preferred embodiment of the application, will be better understood when read in conjunction with the appended drawings. For the purposes of illustrating the device of the present application, there is shown in the drawings a preferred embodiment. It should be understood, however, that the application is not limited to the precise arrangements and instrumentalities shown. In the drawings:

FIG. 1A is a front elevational view of an exemplary embodiment of a bone fixation element and a rod in accordance with a preferred embodiment of the present invention;

FIG. 1B is a cross-sectional view of the bone fixation element and rod shown in FIG. 1A, taken along line 1B-1B of FIG. 2A;

FIG. 2A is a side elevational view of two bone fixation elements supporting the rod which incorporates an optional reduced diameter portion;

FIG. 2B is a cross-sectional view of the bone fixation elements and rod shown in FIG. 2A, taken generally through a center of the rod and into the page of FIG. 2A;

FIG. 3A is an exploded front elevational view of the bone fixation element and rod shown in FIG. 1A;

FIG. 3B is an exploded side elevational view of the bone fixation element and rod shown in FIG. 1A;

FIG. 4A is an exploded top perspective view of the bone fixation element and rod shown in FIG. 1A;

FIG. 4B is a cross-sectional view of the bone fixation element and shown in FIG. 1A, taken along line 4B-4B of FIG. 4A;

FIG. 5A is a top perspective exploded detailed view of first and second rod protectors of the preferred bone fixation element of FIG. 1A; and

FIG. 5B is a top perspective exploded detailed view of the first and second rod protectors shown in FIG. 5A in contact with the rod.

### DETAILED DESCRIPTION OF THE INVENTION

Certain terminology is used in the following description for convenience only and is not limiting. The words “right”, “left”, “lower” and “upper” designate directions in the drawings to which reference is made. The words “inwardly” and “outwardly” refer to directions toward and away from, respectively, the geometric center of the bone fixation element, the rod and designated parts thereof. The words, “anterior”, “posterior”, “superior”, “inferior” and related words and/or phrases designate preferred positions and orientations in the human body to which reference is made and are not meant to be limiting. The terminology includes the above-listed words, derivatives thereof and words of similar import.

A preferred embodiment of the invention will now be described with reference to the drawings. In general, the preferred embodiment relates to a bone fixation element, generally designated 10, for use in posterior spinal fixation to facilitate insertion of a longitudinal spinal rod 45 in a rod-receiving channel formed in the bone fixation element 10. By way of non-limiting example, the spinal rod 45 may be a dynamic spinal rod 45 made from a generally non-biocompatible or less biocompatible material (collectively referred to herein as non-biocompatible). Preferably, the spinal rod 45 is coated to limit direct exposure of the rod 45 to a patient's body. The bone fixation element 10 preferably incorporates first and second rod protectors 120, 140 to help preserve the integrity of the coating on the spinal rod 45 when received in the rod receiving channel of the bone fixation element 10. The bone fixation element 10 and rod 45 may have other applications and uses and should not be limited to the structure or use described and illustrated in the present application.

While the bone fixation element 10 will be described as and may generally be used in the spine (for example, in the lumbar, thoracic or cervical regions), those skilled in the art will appreciate that the bone fixation element 10 may be used for fixation of other parts of the body such as, for example, joints, long bones or bones in the hand, face, feet, extremities, cranium, etc.

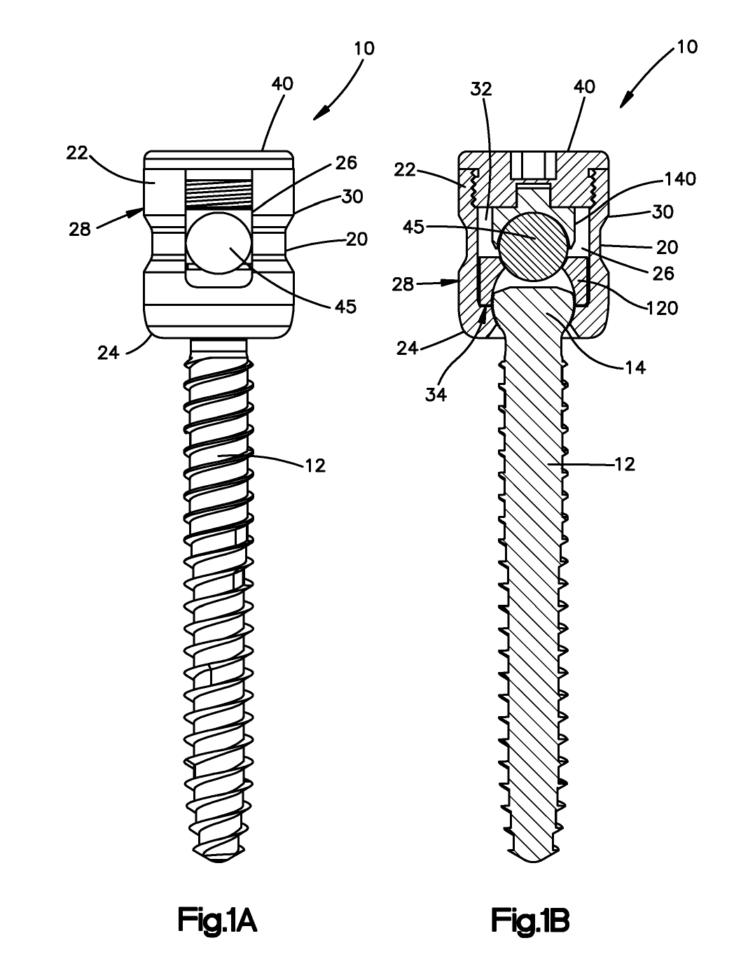

As generally understood by one of ordinary skill in the art, it should be understood that bone fixation element 10 is used generally and may include, but is not limited to, poly-axial or mono-axial pedicle screws, hooks (both mono-axial and poly-axial) including pedicle hooks, transverse process hooks, sublaminar hook, or other fasteners, clamps or implants. Generally speaking, as will be appreciated by one of ordinary skill in the art and as generally shown in FIGS. 1A and 1B, the preferred bone fixation element 10 includes a bone anchor 12 (shown as a bone screw) having an enlarged head portion 14, a body portion 20 (shown as a top loading body portion) having an upper end 22, a lower end 24, and a rod-receiving channel 26 (shown as a top loading U-shaped rod-receiving channel) configured for receiving the spinal rod 45. The rod-receiving channel 26 of the preferred embodiment defines a pair of spaced apart arms 28, 30. The body portion 20 also includes an inner bore 32 extending from the upper end 22 to the lower end 24 and a seat 34 for preventing the enlarged head portion 14 of the bone anchor 12 from passing through the lower end 24 of the body portion 20. The bone fixation element 10 also preferably includes a set screw or closure cap 40, such as, for example, an externally threaded set screw, an internally threaded set screw, a cam lock, a ratchet cap, etc. (collectively referred to herein as a closure cap). As shown and generally described, the enlarged head portion 14 of the bone anchor 12 may be separate from and be disposed within the lower end 24 of the body portion 20 so that the bone anchor 12 can poly-axial rotate with respect to the body portion 20. Alternatively, the bone anchor 12 may be formed integrally with the body portion 20 to form a monolithic structure, which is sometimes referred to as a mono-axial pedicle screw or hook, or if the rod-receiving channel 26 is angled, a fixed angle pedicle screw or hook. Alternatively, the bone fixation element 10 may incorporate a side loading rod-receiving channel.

Once the spinal rod 45 is inserted into the rod-receiving channel 26, the surgeon can secure the position of the spinal rod 45 with respect to the body portion 20 and the position of the bone anchor 12 with respect to the body portion 20 by engaging the closure cap 40. Engagement of the closure cap 40 with the body portion 20 may cause the closure cap 40 to exert a downward force, either directly or indirectly, onto the spinal rod 45. The spinal rod 45 may then exert a downward force, either directly or indirectly, onto the enlarged head portion 14 of the bone anchor 12, thereby securing the position of the bone anchor 12 with respect to the body portion 20 and the position of the rod 45 with respect to the body portion 20.

It should be understood however that the above description is merely exemplary and the present invention is not limited in use to any particular type of bone fixation element. As such, the present invention may be used with other now known or hereafter developed bone fixation elements including, for example, bottom loading bone fixation elements.

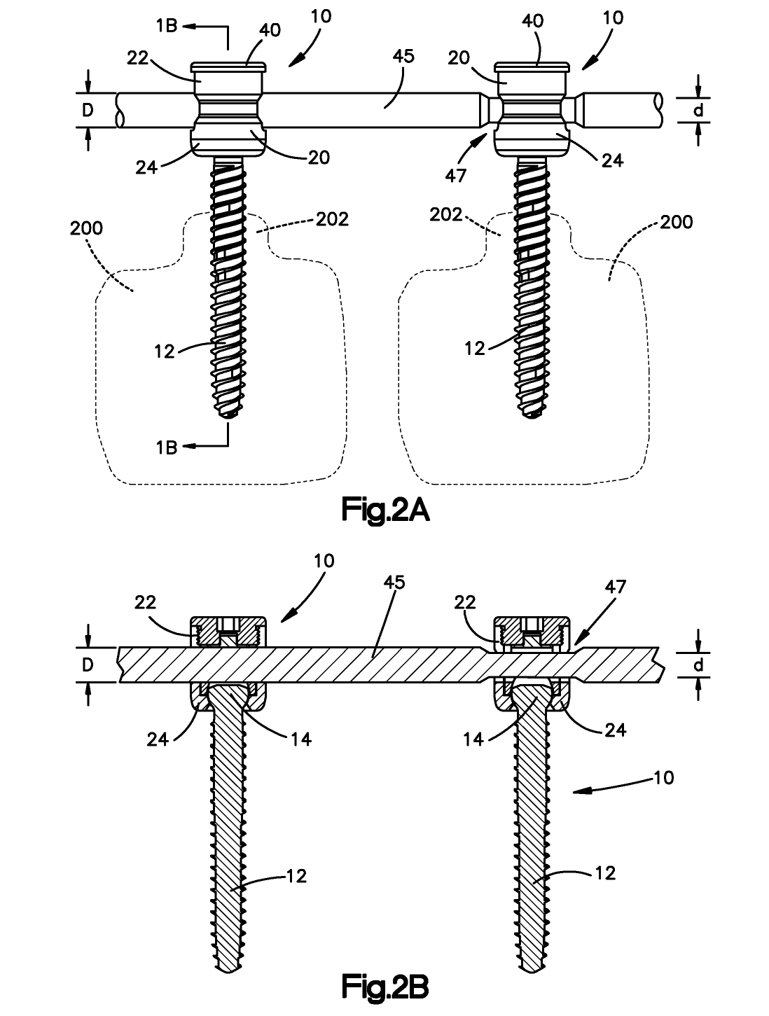

The spinal rod 45 may be manufactured from a traditional biocompatible material, such as, for example, titanium or a titanium alloy. To enhance the bendability of the spinal rod 45, the spinal rod 45 may be manufactured to include a reduced diameter portion 47, which has a smaller diameter d, as best shown in FIGS. 2A and 2B, than a diameter D of the rest of the spinal rod 45. The smaller diameter d of the reduced diameter portion 47 of the spinal rod 45 may be desirable in order to increase the rod's bendability at the reduced diameter portion 47 and may allow the use of smaller bone fixation elements 10. The surfaces of the components in the bone fixation element 10 used to lock the rod 45 may be dimensioned to conform to the shape of the reduced diameter portion 47 of the spinal rod 45. Alternatively, the spinal rod 45 can be manufactured with other now known or hereafter developed characteristics for increasing the rod's bendability such as, for example, the rod 45 can be manufactured with one or more spiral grooves, with one or more holes or tunnels, etc. Alternatively, the spinal rod 45 can be manufactured from numerous components that are configured to couple together while still permitting the rod 45 to bend such as, for example, a ball joint.

Alternatively, the spinal rod 45 may be manufactured from a less traditional material such as, for example, a generally non-biocompatible material. For example, the spinal rod 45 may be manufactured from a material that enables and/or enhances the spinal rod's ability to bend. The spinal rod 45 may be manufactured from, for example, nickel, a nickel alloy, Ni—Ti-alloy (e.g., Nitinol), stainless steel, a memory shaped alloy, cobalt chromium (CoCr) or a cobalt chromium alloy such as, for example, CoCrMo, CoCrMoC, CoCrNi, CoCrWNi, etc.

It is possible that some of these alternative materials may be subject to metal ion diffusion. If a material prone to ion diffusion is used, it may be desirable to prevent or at least reduce release of the ions, since the ions could produce an allergic reaction in the patient's body. For example, if released into the body, nickel, nickel alloy, Nitinol, cobalt chromium, cobalt chromium alloy, may produce an allergic reaction in the body via ion diffusion. The problem of ion diffusion may be reduced by coating the spinal rod 45 with a suitable, preferably bio-compatible material.

However, when a coated spinal rod 45 is inserted into the rod receiving channel 26 of a bone fixation element and then locked in place, the metal components of the bone fixation element can press against and scratch the coating, leaving some of the surface of the rod 45 exposed. It is therefore possible for metal ions to diffuse from the rod 45 through the exposed areas or scratches and produce an allergic reaction in the patient.

It should be understood however that the above description is merely exemplary and the present invention is not limited in use to any particular type of spinal rod. As such, the present invention may be used with any other spinal rod now known or hereafter developed. The present invention however is particularly well suited for use with coated rods, more preferably coated dynamic rods made from a generally non-biocompatible material.

The bone fixation element 10 of the present invention preferably reduces potential ion diffusion and enables the use of generally non-biocompatible materials by providing a structure to protect the rod's coating.

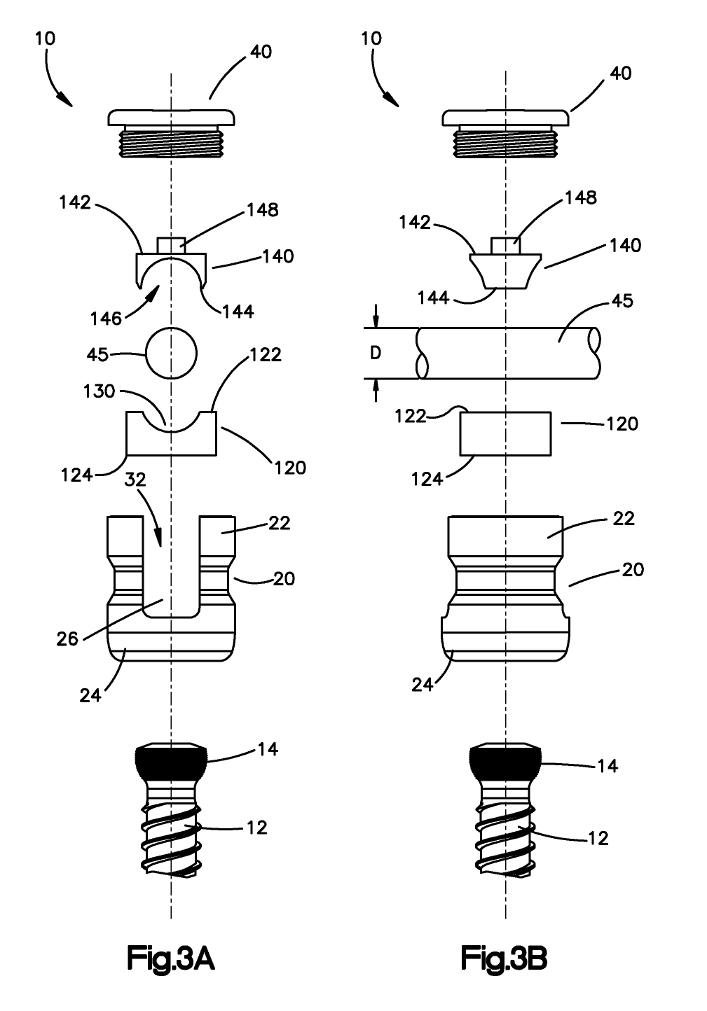

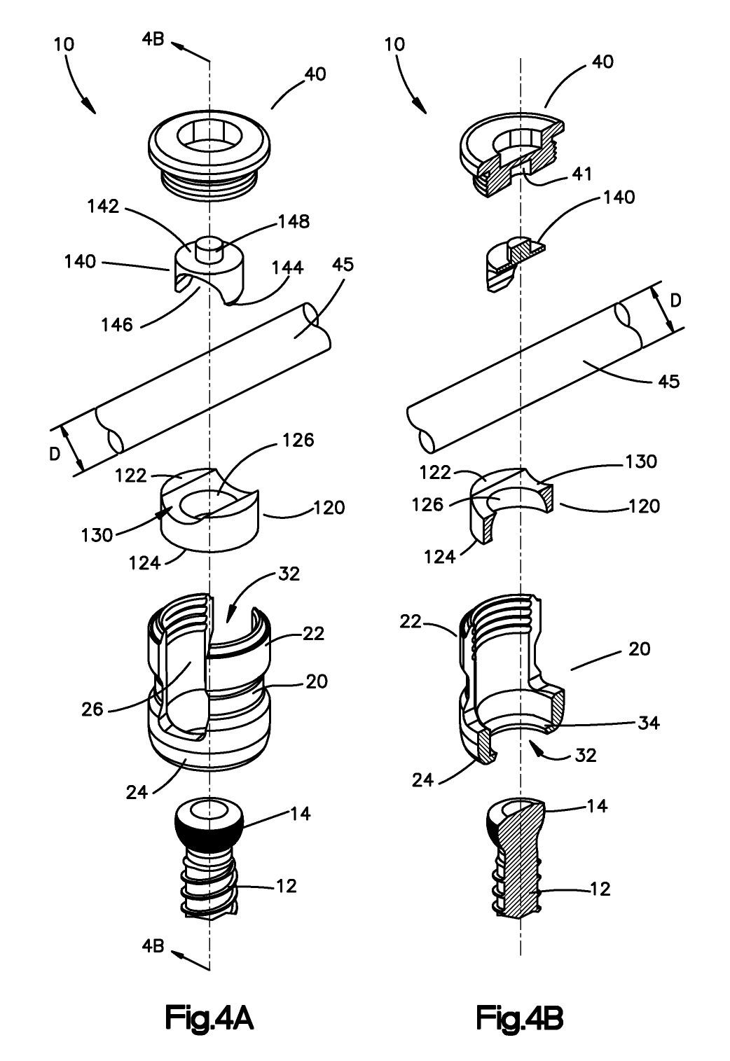

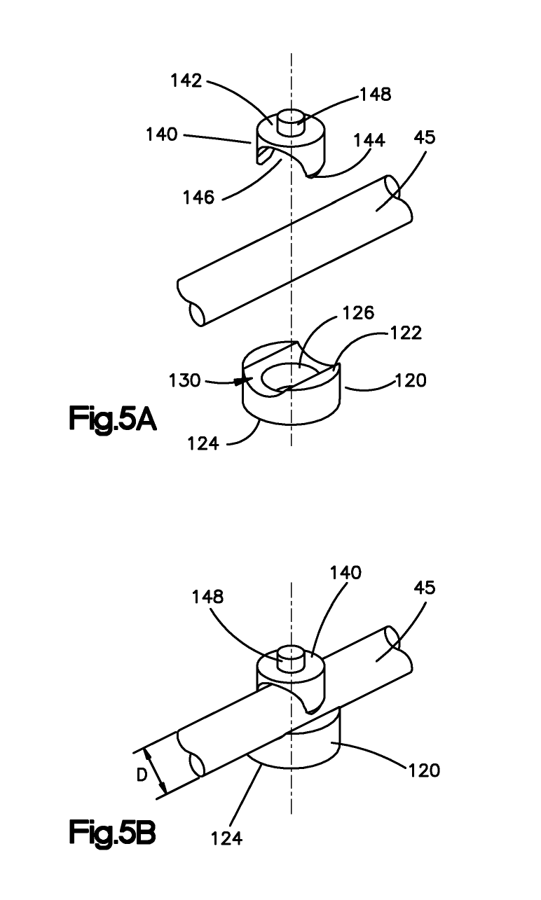

Referring to FIGS. 3A, 3B, 4A, 4B, 5A and 5B, the bone fixation element 10 preferably includes a first rod protector 120 and a second rod protector 140. The first and second rod protectors 120, 140 are preferably internally received within the inner bore 32 of the body portion 20 of the bone fixation element 10. Alternatively, it is contemplated that one or both of the rod protectors 120, 140 can be configured to reside on the outside of the body portion 20 such as, for example, as an outer sleeve. The first rod protector 120 preferably is disposed between the enlarged head portion 14 of the bone anchor 12 and the spinal rod 45 while the second rod protector 140 is preferably disposed between the closure cap 40 and the longitudinal spinal rod 45 so that the first and second rod protectors 120, 140 reside on both sides of the spinal rod 45. Preferably, the first and second rod protectors 120, 140 are configured so that in use, once the closure cap 40 has been fully engaged, the spinal rod 45 is completely surrounded by the first and second rod protectors 120, 140.

The rod protectors 120, 140 are preferably manufactured from a softer, i.e., more elastic material than the material of the longitudinal spinal rod 45. That is, the rod protectors 120, 140 are preferably manufactured from a material having a hardness that is less than the hardness of the spinal rod 45. For example, the rod protectors 120, 140 may be manufactured from a thermoplastic polymer such as polyetheretherketone (PEEK), polyetherketoneketone (PEKK), members of the polyaryletherketone (PEAK) family, polytetrafluoroethylene (PTFE), ultra-high molecular weight polyethylene (UHMWPE), or from a resorbable polymer, which could be amorphous or partially crystalline, such as a resorbable polymer from the poly lactic acid (PLA) family or from the bioresorbable polyurethans such as, for example, polyurtethan urea (PUUR). Alternatively, the rod protectors 120, 140 may be manufactured from a metal such as a titanium alloy comprising molybdenum (TiMo), and appropriate grades of commercially pure titanium (TiCp) such as grade 1 or 2 material, or any other suitable material now known or hereafter developed.

In a particularly preferred embodiment, if the coated spinal rod 45 is made from nickel or a nickel alloy such as Nitinol or a member of the Nitinol family then the first and second rod protectors 120, 140 preferably have a hardness of 0-430 HV 0.5, more preferably 0-380 HV 0.5. Alternatively, if the coated spinal rod 45 is made from cobalt chromium or a cobalt chromium alloy then the first and second rod protectors 120, 140 preferably have a hardness of 0-420 HV 0.5, more preferably 0-400 HV 0.5.

The use of a softer material for manufacturing the rod protectors 120, 140 is preferred because such material generally has better stress shielding ability. That is, owing to the elasticity of the material, the preferred rod protectors 120, 140 are able to deform slightly, which improves the stress distribution or stress shielding ability of the bone fixation element 10. Local stress between components, for example, between the rod protectors 120, 140 and the spinal rod 45, can be reduced because force is distributed over a larger contact area.

As shown, the first rod protector 120 may have a generally cylindrical shape, although other shapes are also envisioned, and generally includes a top surface 122 for contacting the spinal rod 45 and a bottom surface 124 for contacting the enlarged head portion 14 of the bone anchor 12. The first rod protector 120 also preferably includes a bore 126 extending from the top surface 122 to the bottom surface 124 to enable a user to access the enlarged head portion 14 of the bone anchor 12 so that, for example, the bone anchor 12 can be rotated via a screwdriver. The bottom surface 124 may include a curvate surface (not shown) for contacting at least a portion of the enlarged head portion 14 of the bone anchor 12. Alternatively, the bottom surface 124 may include an inner cavity (not shown) for receiving at least a portion of the enlarged head portion 14 of the bone anchor 12. The top surface 122 of the first rod protector 120 preferably includes a saddle 130 for contacting and/or receiving at least a portion of the spinal rod 45.

Referring to FIGS. 3A-5B, the second rod protector 140 preferably includes a top surface 142 and a bottom surface 144, wherein the bottom surface 144 preferably includes a saddle 146 for contacting and/or receiving at least a portion of the spinal rod 45. The second rod protector 140 may be coupled to the closure cap 40 by any means now known or hereafter developed for such purpose. For example, the second rod protector 140 preferably includes a stem 148 projecting upwards from the top surface 142, wherein the stem 148 is receivable within a bore 41 formed in the closure cap 40. The second rod protector 140 is coupled to the closure cap 40, but preferably is free to rotate with respect to the closure cap 40 so that the saddle 146 formed in the bottom surface 144 of the second rod protector 140 can self-align with the rod 140 and the saddle 146 may engage the rod 45 while the closure cap 40 is rotated to tighten or loosen the closure cap 40 relative to the body portion 20.

The top surface 142 of the second rod protector 140 preferably is configured to contact and receive forces from the bottom surface of the closure cap 40. If the contacting surfaces have the proper shape, the pressure levels generated by the applied force can be controlled. In particular, as shown, the top surface 142 of the second rod protector 140 preferably includes a flat surface against which the bottom surface of the closure cap 40 can be pressed.

Preferably, the saddles 130, 146 formed in the top surface 122 of the first rod protector 120 and the bottom surface 144 of the second rod protector 140, respectively, are shaped to correspond to the outer surface of the rod 45. That is, the saddles 130, 146 preferably have a radius of curvature about the same as the radius of curvature of the spinal rod 45. In this manner, any force between the rod 45 and the first and second rod protectors 120, 140 will be well-distributed, and damage to the coating on the surface of the rod 45 can be limited. Moreover, as previously mentioned, the first and second rod protectors 120, 140 are preferably configured so that in use, once the closure cap 40 has been fully engaged, the spinal rod 45 is completely surrounded by the first and second rod protectors 120, 140, thus further helping to limit damage to the coating on the surface of the rod 45. Such force, it will be appreciated, can arise during implantation of the bone fixation element 10, engagement with the rod 45, and/or while implanted during bending, extension, compression or twisting of the patient's spine.

It should be understood however that the above description of the shape of the first and second rod protectors 120, 140 are merely exemplary and the first and second rod protectors 120, 140 are not limited to any particular shape. As such, the first and second rod protectors 120, 140 may take on other shapes. Moreover, it will be appreciated that the first and second rod protectors 120, 140 can be designed with sizes and shapes chosen to facilitate the ability of the protectors 120, 140 to work with a particular sized and shaped rod 45 and/or a particular sized and shaped bone anchor 12.

Referring to FIGS. 1-5B, in use, to assemble the bone fixation element 10, the rod 45 is received within the rod receiving channel 26 of the bone fixation element 10 on top of the first rod protector 120. If the first rod protector 120 is able to rotate relative to the body portion 20, it may be necessary to rotate the first rod protector 120 so that the saddle 130 formed in the top surface 122 of the first rod protector 120 is aligned with the rod receiving channel 26, alternatively an alignment mechanism such as, for example, a tab may be incorporated to self align the saddle 130 with the rod receiving channel 26 or the rod protector 120 may be fixed to or integral with the body portion 20 and pre-aligned in a preferred orientation. Next, the second rod protector 140 is placed on top of the rod 140 such that the rod 45 fits into the saddle 146 formed in the bottom surface 144 of the second rod protector 140. The bone anchor 12 is then preferably implanted into a vertebral body 200, preferably through a pedicle 202 to secure the bone anchor 12 and body portion 20 to the vertebra 200. The closure cap 40 is then placed into engagement with the body portion 20 of the bone fixation element 10 to close the bore 32 formed in the body portion 20 and the saddle 146 engages the rod 45. Engagement of the closure cap 40 may cause the closure cap 40 to apply a downward force onto the second rod protector 140, which in turn may apply a downward force onto the spinal rod 45 and the first rod protector 120, thereby securing the position of the rod 45 relative to the body portion 20. Also, if the first rod protector 120 is configured to press against the enlarged head portion 14 of the bone anchor 12, the downward force may cause the first rod protector 120 to press against the enlarged head portion 14, which in turn may cause the enlarged head portion 14 to press against the seat 34 formed in the body portion 20, thereby securing the position of the body portion 20 with respect to the bone anchor 12.

While the foregoing embodiment involves the use of two rod protectors 120, 140, this invention is not limited to such an arrangement. Alternative designs could employ one, three or even more rod protectors (not shown). Assembly techniques will vary depending upon the number of rod protectors that are used.

As will be readily appreciated by one of ordinary skill in the art, in use, spinal stabilization may take on several different methodologies for multi-segmental treatment such as, for example, full fixation for posterolateral fusion, combined fixation and stabilization where the fused segments receive a stabilized segment on top in order to dampen the motion above the fused segments, full stabilization for stress reduction for example in elderly patients, or hybrid fixation where the lower segments of the spine are stabilized with dampening means, such as, for example, a dynamic spinal rod and stabilization which becomes mobile again. Thus, for example, one may incorporate the polymeric resorbable rod protectors 120, 140 to enable further mobilization after resorption of the rod protectors 120, 140. That is, in order to regain mobility, one vertebra 200 may be secured by a bone fixation element 10 incorporating, for example, first and second rod protectors 120, 140 made from a thermoplastic polymer or metal, while subsequent vertebrae 200 may be secured by a bone fixation element 10 incorporating, for example, resorbable polymers so that the patient can be remobilized once the resorbable rod protectors 120, 140 have been absorbed.

Although the present invention may be of particular benefit when used with rods made from a generally non-biocompatible material such that it is beneficial to coat the spinal rod 45 with a biocompatible material, the present invention is not limited thereto. The preferred embodiment of the bone fixation element 10 also can be used with coated rods 45 of highly biocompatible material such as, for example, titanium or titanium alloy. The preferred embodiment can also be used with rods 45 made from any other material now known or hereafter developed, and biocompatible coatings now known or hereafter developed.

It will be appreciated by those skilled in the art that changes could be made to the embodiment described above without departing from the broad inventive concept thereof. It is understood, therefore, that this invention is not limited to the particular embodiment disclosed, but it is intended to cover modifications within the spirit and scope of the present invention as defined by the appended claims.

1-22. (canceled)

23. A bone fixation system for mounting to vertebrae, the bone fixation system comprising:
a coated, spinal rod; and
at least two bone fixation elements, each bone fixation element comprising:
a bone anchor;
a body portion having an inner bore and a rod-receiving channel dimensioned to receive the coated spinal rod;
a first rod protector dimensioned to fit within the inner bore of the body portion, the first rod protector having a top surface for contacting the coated spinal rod;
a second rod protector dimensioned to fit within the inner bore of the body, the second rod protector having a bottom surface for contacting the coated spinal rod; and
a closure cap configured to engage the body portion for at least partially obstructing the rod receiving channel to secure the coated spinal rod in the rod-receiving channel of the body portion, wherein the first and second rod protectors are constructed of a material having a hardness less than a hardness of a material of the coated spinal rod;
wherein the first and second rod protectors are manufactured from a material selected from the group consisting of polyetheretherketone (PEEK), polyetherketoneketone (PEKK), polyaryletherketone (PEAK), polytetrafluoroethylene (PTFE), ultra-high molecular weight polyethylene (UHMWPE), poly lactic acid (PLA), and polyurtethan urea (PUUR).

24. The bone fixation system of claim 23, wherein the coated spinal rod is a dynamic spinal rod such that the rod is bendable to facilitate movement of adjacent vertebrae.

25. The bone fixation system of claim 24, wherein at least a portion of the coated spinal rod includes a reduced diameter portion

26. The bone fixation system of claim 23, wherein the coated spinal rod is made from a non-biocompatible material.

27. The bone fixation system of claim 26, wherein the material of the coated spinal rod is selected from the group consisting of Nitinol, a Nitinol alloy, cobalt chromium, a cobalt chromium alloy, stainless steel, and a shape memory alloy.

28. The bone fixation system of claim 26, wherein the material of the coated spinal rod is selected from the group consisting of nickel and a nickel alloy.

29. The bone fixation system of claim 23, wherein the material of the coated spinal rod is constructed of Nitinol or a member of the Nitinol family and the material of the first and second rod protectors has a hardness of 0-430 HV 0.5.

30. The bone fixation system of claim 23, wherein the material of the coated spinal rod is constructed of a Nitinol or a member of the Nitinol family and the material of the first and second rod protectors has a hardness of 0-380 HV 0.5.

31. The bone fixation system of claim 23, wherein the material of the coated spinal rod is constructed of a cobalt chromium or a cobalt chromium alloy and the material of the first and second rod protectors has a hardness of 0-420 HV 0.5.

32. The bone fixation system of claim 23, wherein the material of the coated spinal rod is constructed of a cobalt chromium or a cobalt chromium alloy and the material of the first and second rod protectors has a hardness of 0-400 HV 0.5.

33. The bone fixation system of claim 23, wherein the bone anchor is poly-axially rotatable with respect to the body portion.

34. The bone fixation system of claim 33, wherein the first rod protector is disposed between an enlarged head portion of the bone anchor and the coated spinal rod when the spinal rod is received within the rod receiving channel and the second rod protector is disposed between the closure cap and the coated spinal rod when the spinal rod is received within the rod receiving channel and the closure cap engages the body portion.

35. The bone fixation system of claim 33, wherein the first and second rod protectors are configured to substantially surround the coated spinal rod once the closure cap is engaged with the body portion.

36. The bone fixation system of claim 23, wherein the material of the first and second rod protectors has a hardness A and the material of the coated spinal rod has a hardness B, the hardness A being less than the hardness B.

37. The bone fixation system of claim 36, wherein the first and second rod protectors are configured to deform about the spinal rod as the closure cap is engaged to the body portion.

38. The bone fixation system of claim 37, wherein the top surface of the first rod protector and the bottom surface of the second rod protector include a saddle having a radius of curvate configured to substantially correspond with a radius of curvature of the coated spinal rod.

39. A bone fixation system for mounting to vertebrae, the bone fixation system comprising:
a coated, spinal rod; and
at least two bone fixation elements, each bone fixation element comprising:
a bone anchor;
a body portion having an inner bore and a rod-receiving channel dimensioned to receive the coated spinal rod;
a first rod protector dimensioned to fit within the inner bore of the body portion, the first rod protector having a top surface for contacting the coated spinal rod;
a second rod protector dimensioned to fit within the inner bore of the body, the second rod protector having a bottom surface for contacting the coated spinal rod; and
a closure cap configured to engage the body portion for at least partially obstructing the rod receiving channel to secure the coated spinal rod in the rod-receiving channel of the body portion, wherein the first and second rod protectors are constructed of a material having a hardness less than a hardness of a material of the coated spinal rod;
wherein the first and second rod protectors are manufactured from a material having a hardness A and the spinal rod is manufactured from a material having a hardness B, the hardness A of the first and second rod protectors being less than the hardness B of the spinal rod.

40. The bone fixation system of claim 39, wherein the material of the coated spinal rod is constructed of Nitinol or a member of the Nitinol family and the material of the first and second rod protectors has a hardness of 0-430 HV 0.5.

41. The bone fixation system of claim 39, wherein the material of the coated spinal rod is constructed of a Nitinol or a member of the Nitinol family and the material of the first and second rod protectors has a hardness of 0-380 HV 0.5.

42. The bone fixation system of claim 39, wherein the material of the coated spinal rod is constructed of a cobalt chromium or a cobalt chromium alloy and the material of the first and second rod protectors has a hardness of 0-420 HV 0.5.

43. The bone fixation system of claim 39, wherein the material of the coated spinal rod is constructed of a cobalt chromium or a cobalt chromium alloy and the material of the first and second rod protectors has a hardness of 0-400 HV 0.5.
